(19) United States
(12) Patent Application Publication
(10) Pub. No.: US 2008/0294205 A1
(43) Pub. Date: Nov. 27, 2008

(54) EXPANDABLE SUPPORT DEVICE AND METHOD OF USE

(75) Inventors: E. Skott Greenhalgh, Lower Gwynedd, PA (US); John-Paul Romano, Chalfont, PA (US)

Correspondence Address: LEVINE BAGADE HAN LLP, 2483 EAST BAYSHORE ROAD, SUITE 100, PALO ALTO, CA, 94303, US

(73) Assignee: Stout Medical Group, L.P., Perkasie, PA (US)

(21) Appl. No.: 12/139,396

(22) Filed: Jun. 13, 2008

Related U.S. Application Data
(63) Continuation of application No. PCT/US2006/062201, filed on Dec. 15, 2006.
(60) Provisional application No. 60/751,390, filed on Dec. 15, 2005.

(51) Int. Cl. A61F 5/00; A61M 29/00

(52) U.S. Cl. 606/86 A; 606/191

(57) ABSTRACT

A deployment system and a method of using the deployment system are disclosed. The deployment system can have an expandable support device that can be used to treat orthopedic injuries. The expandable support device can be deployed with or between bones. The deployment system can be integral with the expandable support device. The deployment system can be designed to release the expandable support device when a specific deployment force is exerted onto the deployment system.

### CROSS-REFERENCE TO RELATED APPLICATIONS

This application is a continuation of PCT International Application No. PCT/US2006/062201, filed Dec. 15, 2006 which claims the benefit of U.S. Provisional Application No. 60/751,390, filed Dec. 15, 2005, which are both incorporated herein in their entireties.

### BACKGROUND OF THE INVENTION

This invention relates to devices and methods for holding and deploying orthopedic and other expandable support devices (e.g., stents). The expandable support devices can be used for providing support for biological tissue, for example to repair spinal compression fractures.

When performing any medical operation with an implant the implant must be delivered to the treatment site and the implant device must be properly designed and deployed. Important implant device design and deployment characteristics include size, shape, function, material, mechanical properties, and chemical properties, among others.

Vertebroplasty is an image-guided, minimally invasive, nonsurgical therapy used to strengthen a broken vertebra that has been weakened by disease, such as osteoporosis or cancer. Vertebroplasty is often used to treat compression fractures, such as those caused by osteoporosis, cancer, or stress.

Vertebroplasty is often performed on patients too elderly or frail to tolerate open spinal surgery, or with bones too weak for surgical spinal repair. Patients with vertebral damage due to a malignant tumor may sometimes benefit from vertebroplasty. The procedure can also be used in younger patients whose osteoporosis is caused by long-term steroid treatment or a metabolic disorder.

Vertebroplasty can increase the patient's functional abilities, allow a return to the previous level of activity, and prevent further vertebral collapse. Vertebroplasty attempts to also alleviate the pain caused by a compression fracture.

Vertebroplasty is often accomplished by injecting an orthopedic cement mixture through a needle into the fractured bone. The cement mixture can leak from the bone, potentially entering a dangerous location such as the spinal canal. The cement mixture, which is naturally viscous, is difficult to inject through small diameter needles, and thus many practitioners choose to “thin out” the cement mixture to improve cement injection, which ultimately exacerbates the leakage problems. The flow of the cement liquid also naturally follows the path of least resistance once it enters the bone—naturally along the cracks formed during the compression fracture. This further exacerbates the leakage.

The mixture also fills or substantially fills the cavity of the compression fracture and is limited to certain chemical composition, thereby limiting the amount of otherwise beneficial compounds that can be added to the fracture zone to improve healing. Further, a balloon must first be inserted in the compression fracture and the vertebra must be expanded before the cement is injected into the newly formed space.

A vertebroplasty device and method that eliminates or reduces the risks and complexity of the existing art is desired. An easily deployed orthopedic expandable support device that can be controllably delivered and deployed is desired. Being able to recapture the orthopedic expandable support device is also desired.

### BRIEF SUMMARY OF THE INVENTION

A deployment system that can include an expandable support device for performing completely implantable spinal repair is disclosed. The expandable support device can be self-expanding. The expandable support device can be deformably expanded by external forces.

The expandable support device can have a first (e.g., distal) end and a second (e.g., proximal) end. The deployment system can control one or both of the first and second ends of the expandable support device until the expandable support device is substantially or completely deployed in a treatment site.

The deployment system can have a threaded rod. The threaded rod can threadably attach to the expandable support device. A compression force can be delivered (e.g., in part) by the rod to the first end of the expandable support device. The threaded rod can release the expandable support device from the remainder of the deployment system, for example by rotating the rod relative to the expandable support device (e.g., unscrewing the rod from the expandable support device). The rod can be reattached (e.g., by screwing) to the expandable support device. The expandable support device can then be repositioned.

The deployment system can have a rod that can have a rod head (e.g., paddle) that can extend through and beyond a distal port in the first end of the expandable support device. The rod head can be larger that the distal port in a first dimension. The rod head can be smaller than the distal port in a second dimension. In a first configuration, the rod head can be interference fit to the first end of the expandable support device. The rod can be rotatable within the distal port. The rod head can deliver a compression force to the first end of the expandable support device. The rod head can engage the expandable support device on one, two or more (e.g., across the entire rod head) points on the first end of the expandable support device.

After the expandable support device is radially expanded using a compressive force, the rod head can be rotated (e.g., about 90 degrees) relative to the expandable support device. The rod can be translated through the expandable support device, removing the rod head. The rod can have a non-round configuration. The non-round rod can guide radial expansion of the expandable support device (e.g., by transmitting torque from the rod to the distal end port's inner walls). Once the stent expandable support device is expanded, the rod and rod head can be turned 90 degrees and the rod's diameter is decreases to release the rod from the stents inside walls.

The deployment system can have a rod that can have a wedge-shaped rod head. The rod head can be retractable into the rod, for example to withdraw the rod through the distal port in the expandable support device. The retraction of the rod head can be resisted by a spring, for example, to prevent retraction of the rod head before deployment. The rod head retraction can be remotely (e.g., mechanically or electrically) controlled.

The deployment system can be covered by a sheath. The sheath can constrain radial expansion of the expandable support device. The sheath can slide or otherwise translate off of the expandable support device and/or a pusher or driver can force the expandable support device out of the open end of the sheath. The sheath can self-expand and/or be deformably expanded once completely or partially out of the sheath.

The deployment system can have a rod that can have a pin. The rod can have a pin attached to, and extending radially from, the rod. In pre-deployment and compression configurations, the pin can be constrained by the expandable support device. Once the expandable support device is radially expanded, the pin and/or expandable support device can deform out of the constrained configuration and/or the ends of the pin can shear off, detaching the expandable support device and the deployment system.

A method for repairing a damaged section of a spine is also disclosed. The method includes expanding the expandable support device in the damaged section.

### BRIEF DESCRIPTION OF THE DRAWINGS

FIG. 1 is a perspective view of an embodiment of the deployment system in a first configuration.

FIG. 2a illustrates an embodiment of cross-section A-A of FIG. 1.

FIG. 2b illustrates an embodiment of cross-section A1-A1 of FIG. 2a.

FIG. 3 is a perspective view of the deployment system of FIG. 1 in a second configuration.

FIG. 4 illustrates an embodiment of cross-section B-B of FIG. 3.

FIG. 5 is a perspective view of the deployment system of FIG. 1 in a third configuration.

FIG. 6a illustrates an embodiment of cross-section C-C of FIG. 5.

FIG. 6b illustrates an embodiment of cross-section C1-C1 of FIG. 6a.

FIG. 7 is a perspective view of an embodiment of the deployment system.

FIGS. 8 through 10 illustrate various embodiments of cross-section D-D of FIG. 7.

FIG. 11 is a front orthogonal view of an embodiment of the expandable support device.

FIG. 12 is a front orthogonal view of an embodiment of the rod.

FIG. 13 is a front orthogonal view of an embodiment of the deployment system in a first configuration.

FIG. 14 is a front view of the deployment system of FIG. 13.

FIG. 15 is a side view of an embodiment of the deployment system in a first configuration.

FIG. 16 is a top view of the deployment system of FIG. 15.

FIG. 17 is a front view of the deployment system of FIG. 15.

FIG. 18 is a front orthogonal view of the embodiment of the deployment system of FIG. 13 in a second configuration.

FIG. 19 is a front view of the deployment system of FIG. 18.

FIG. 20 is a side view of the deployment system of FIG. 15 in a second configuration.

FIG. 21 is a top view of the deployment system of FIG. 20.

FIG. 22 is a front view of the deployment system of FIG. 20.

FIG. 23 is a front orthogonal view of the embodiment of the deployment system of FIG. 13 in a third configuration.

FIG. 24 is a front view of the deployment system of FIG. 23.

FIG. 25 is a side view of the deployment system of FIG. 15 in a third configuration.

FIG. 26 is a top view of the deployment system of FIG. 25.

FIG. 27 is a front view of the deployment system of FIG. 25.

FIG. 28 is a front orthogonal view of the embodiment of the deployment system of FIG. 13 in a fourth configuration.

FIG. 29 is a front view of the deployment system of FIG. 28.

FIGS. 30 through 32 illustrate an embodiment of a rod in various configurations.

FIGS. 33 through 35 illustrate an embodiment of a rod in various configurations.

FIGS. 36 through 38 illustrate an embodiment of the deployment system in various configurations.

FIGS. 39 and 40 illustrate various embodiments of a cross-section of the deployment system.

FIGS. 41 and 42 illustrate various configurations of the cross-section of the embodiment of the deployment system of FIG. 39 during an embodiment of a method of use.

FIGS. 43 and 44 illustrate various embodiments of a cross-section of the deployment system.

FIGS. 45 through 48 illustrate various configurations of the cross-section of the embodiment of the deployment system of FIG. 44 during various embodiments of methods of use.

FIG. 49 illustrates variations of methods for using a variation of the deployment system in anatomical structure.

FIGS. 50, and 52 through 54 illustrate a variation of a method for using a variation of the deployment system.

FIG. 51 is a close-up view of section E of a variation of the configuration of the variation of the deployment system of FIG. 50.

FIGS. 55, and 56 through 61 illustrate a variation of a method for using a variation of the deployment system.

FIG. 56 is a close-up view of section F of a configuration of the variation of the deployment system of FIG. 55.

### DETAILED DESCRIPTION

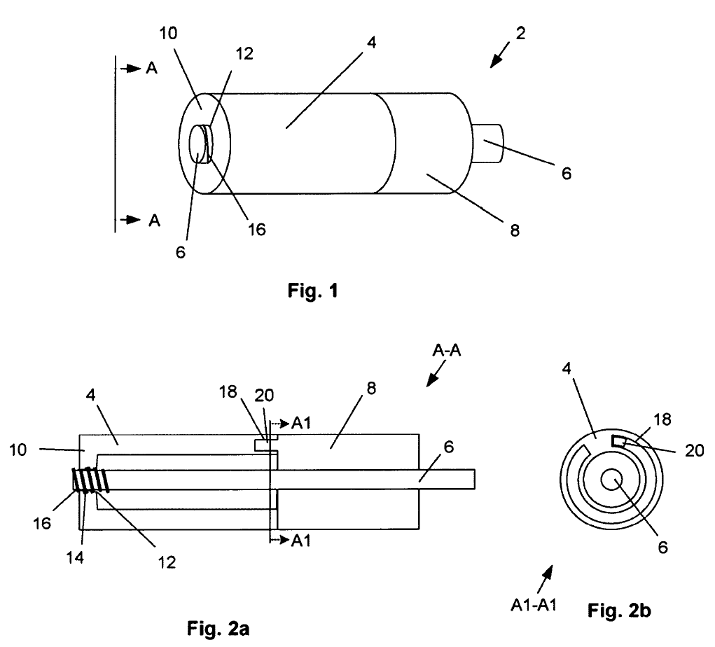

FIGS. 1, 2a and 2b illustrate a deployment system 2 that can have an expandable support device 4. The expandable support device 4 can be used, for example, as an orthopedic support device. The expandable support device 4 can be deployed, for example, between and/or within bones, such as deployment in or around the vertebra (e.g., intravertebral and/or intervertebral), phalanges, tarsals, clavicle, or other bones. The deployment system 2 can be used to treat damage to bones from trauma, disease, or combinations thereof. The deployment system 2 can have a first, longitudinally uncompressed, configuration.

The expandable support device 4 can be releasably attached to a compression apparatus, for example a rod 6 translatably attached (e.g., slidably or threadedly attached) to an anvil 8. The expandable support device 4 can have a compression and/or tensile interference fit with the anvil 8. The expandable support device 4 can releasably attach to the rod 6. The rod 6 can be internal to the anvil 8. The rod 6 can pass through the center of the anvil 8. The rod 6 can pass through a side of the anvil 8. The rod 6 can be external to the anvil 8.

The expandable support device 4 can have a device distal end 10. The device distal end 10 can have a distal end port 12. The distal end port 12 can have a circular configuration. The distal end port 12 can be releasably attached to the rod 6. The distal end port 12 can have device threads 14. The device threads 14 can be integral with the distal end port 12. All or a portion of the rod 6 can have rod threads 16. The rod threads can be integral with the rod 6. The rod threads 16 can threadably attach with the device threads 14.

The expandable support device 4 can have a key slot 18. The anvil 8 can have a key 20. The key slot 18 can slidably attach with the key 20. The key slot 18 can extend less than 360 degrees around the expandable support device 4.

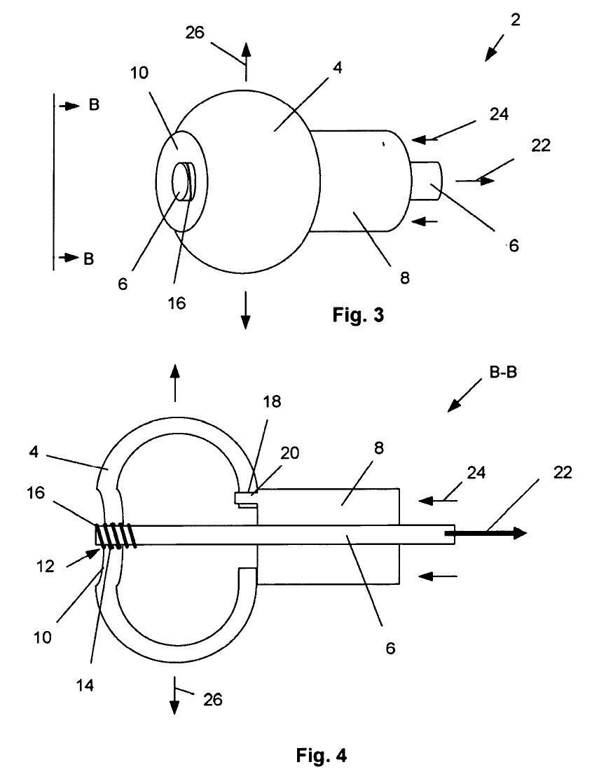

FIGS. 3 and 4 illustrate that a rod compression force 22, as shown by arrow, and an anvil compression force 24, as shown by arrows, can be applied to the deployment system 2. The deployment system 2 can have a second, longitudinally compressed, configuration. The device distal end 10 can be forced toward the anvil 8. The expandable support device 4 can be longitudinally compressed between the rod thread 16 and the anvil 8. The expandable support device 4 can radially expand 26, as shown by arrows.

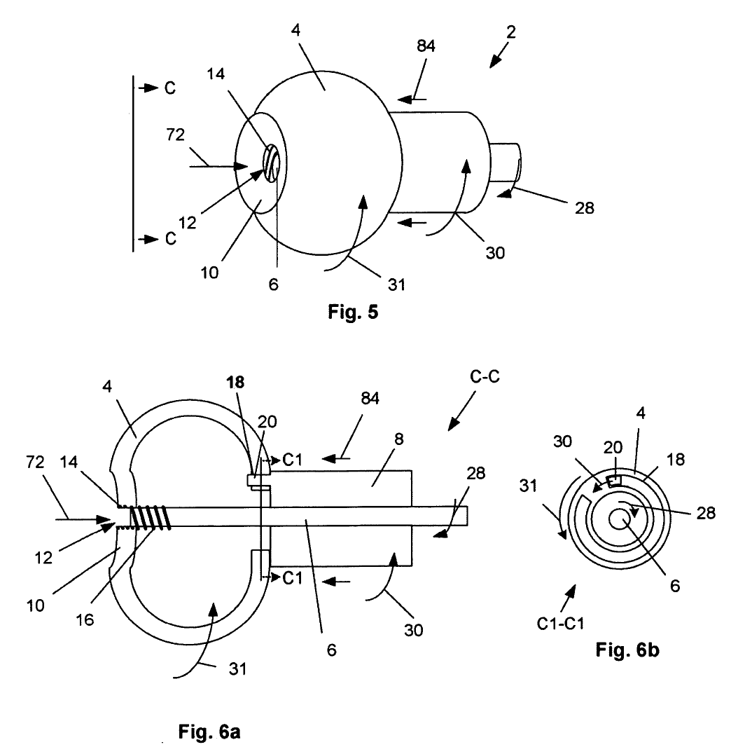

FIGS. 5, 6a and 6b illustrate that a rod rotation 28, as shown by arrow, and an anvil rotation 30, as shown by arrow, directed oppositely to the rod rotation 28 can be imparted on the rod 6 and the anvil 8, respectively. The key 20 can abut a terminus of the key slot 18. The key 20 can transmit the rotational force (i.e., torque) from the anvil 8 to the expandable support device 4. The expandable support device 4 can rotate in synchronicity with the anvil 8.

The rotation of the anvil 30 and the expandable support device 4 are relative to the rod 6. The rotation of the rod 28 is relative to the anvil 8 and the expandable support device 4.

The rod 6 can detach from the expandable support device 4. The rod thread 16 can unscrew from the device thread 14, for example as the expandable support device 4 rotates with respect to the expandable support device 4. The anvil 8 can be translatably detached (not shown) from the expandable support device 4. The expandable support device 4 can be deployed.

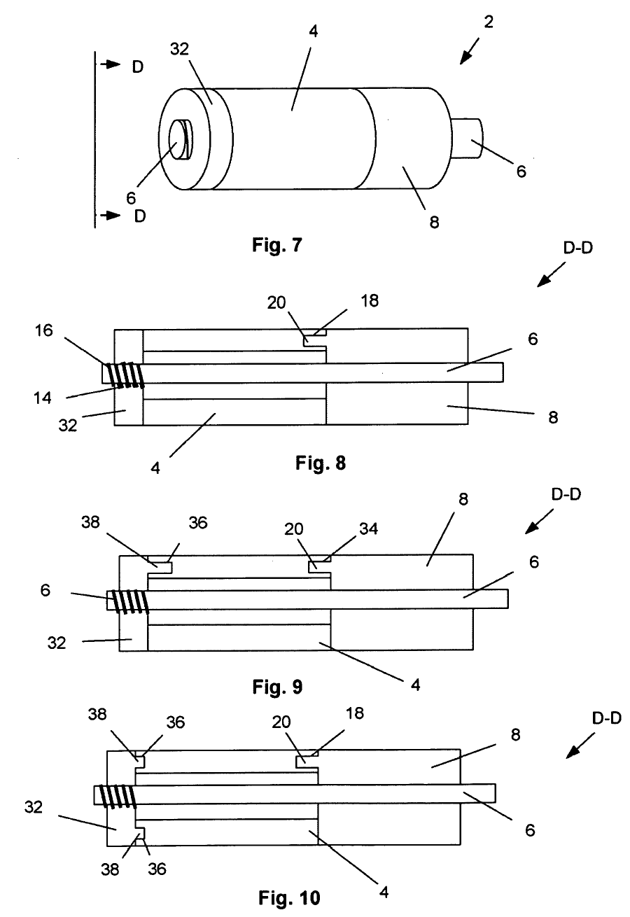

FIGS. 7 through 10 illustrate that the deployment system 2 can have a cap 32. The cap 32 can be removably attached to the distal end of the expandable support device 4. The cap 32 can be removably attached to the expandable support device 4. The cap 32 can releasably attach with the expandable support device 4. For example, the cap 32 can 9 attach to the expandable support device 4 via threads, abutting, snaps, adhesive, one or more grooves, one or more hook and loop attachments, biodegradable suturing, or combinations thereof.

As shown in FIG. 8, the expandable support device 4 can have a key slot 18. As shown in FIG. 9, the expandable a first key slot 34 and a second key slot 36. The first key slot 34 can be adjacent to the anvil 8. The second key slot 36 can be adjacent to the cap 32. As shown in FIG. 9, the cap 32 can have a second key 38. As shown in FIG. 10, the cap 32 can have two second keys 38 and/or a single second key 38 that covers more than or equal to about 180 degrees around the cap 32. The anvil 8 can have multiple keys (not shown), and/or a single key 20 that covers more than or equal to about 180 degrees around the anvil 8.

The keys 20 and key slots 18 can be reversed (i.e., each key slot 18 can be a key 20 and each key 20 can be a key slot 18).

The remainder of the deployment system 2 (e.g., a threaded rod) can recapture or reattach to the expandable support device 4 after the expandable support device 4 has separated from the remainder of the deployment system 2. For example, the rod 6 can be threaded into the distal end port 12. The reattachment can occur at any time after detachment, including after the expandable support device 4 is radially expanded 26. The expandable support device 4 can be radially contracted by the remainder of the deployment system 2 before, during or after radial expansion 26 of the expandable support device 4.

FIG. 11 illustrates that the distal end port 12 of the expandable support device 4 can have a rectangular (as shown), square, oval, triangular, pentagonal configuration, or combinations thereof. The distal end port 12 can have a port first axis 40 and a port second axis 42. The distal end port 12 can have additional port axes (not shown), for example for triangular or pentagonal configurations. The distal end port 12 can have a port height 44 parallel with the port first axis 40. The distal end port 12 can have a port width 46 parallel with the port second axis 42.

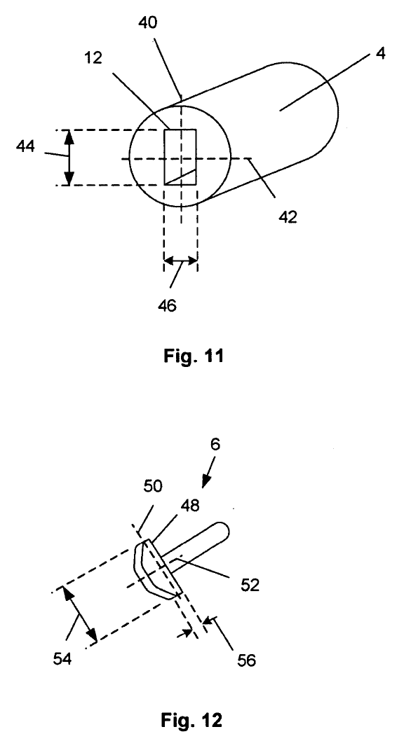

FIG. 12 illustrates that the rod 6 can have a rod head 48 or paddle. The rod head can be radially larger than the remainder of the rod 6 and/or the rod 6 adjacent to the rod head 48. The rod head 48 can have a rectangular (as shown), square, oval, triangular, pentagonal configuration, or combinations thereof. The rod head 48 can have a head first axis 50 and a head second axis 52. The rod head 48 can have additional head axes (not shown), for example for triangular or pentagonal configurations. The rod head 48 can have a head height 54 parallel with the head first axis 50. The rod head 48 can have a head width 56 parallel with the head second axis 52.

The head width 56 can be smaller than the port width 46. The head height 54 can be smaller than the port height 44. The head height 54 can be larger than the port width or the head width 56 can be larger than the port height 44.

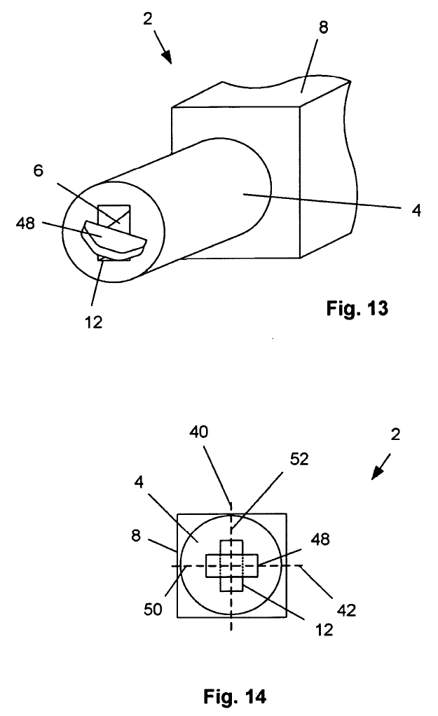

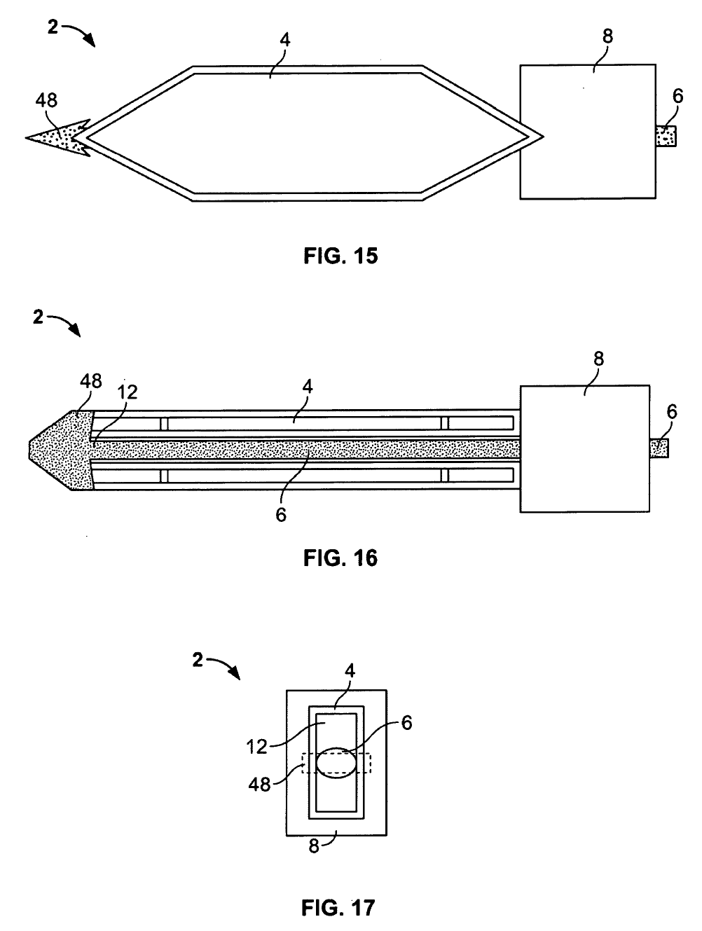

FIGS. 13 and 14 and separately FIGS. 15 through 17 illustrate various embodiments of the deployment system 2 that can be in a first, longitudinally uncompressed configuration. The rod 6 can extend out of the distal end port 12. The rod head 48 can be outside of the expandable support device 4, for example, beyond the distal end port 12. The rod 6 can be rotated relative to the expandable support device 4, for example, so that the port second axis 42 is substantially or completely aligned with the head first axis 50. The rod 6 can be rotated relative to the expandable support device 4, for example, so that the port first axis 40 is substantially or completely aligned with the head second axis 52. In the first (e.g., pre-compression and compression) configuration, the rod head 48 can interference fit with the expandable support device 4.

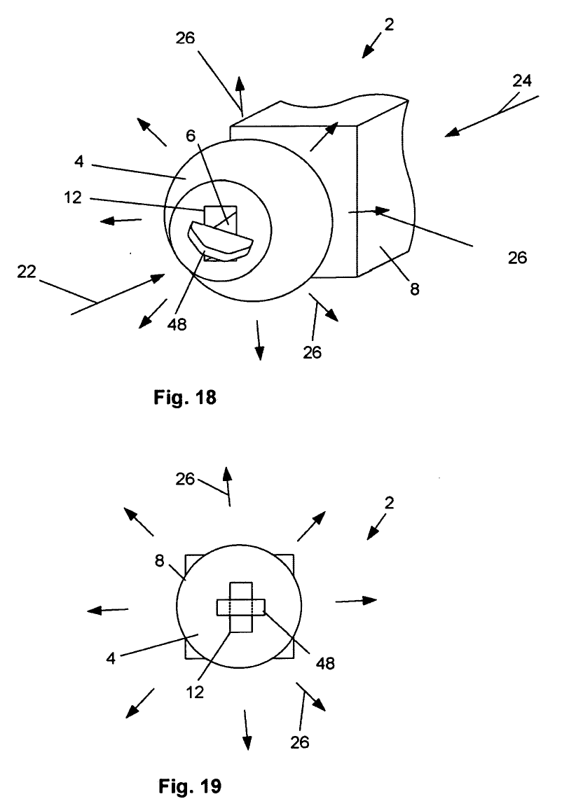

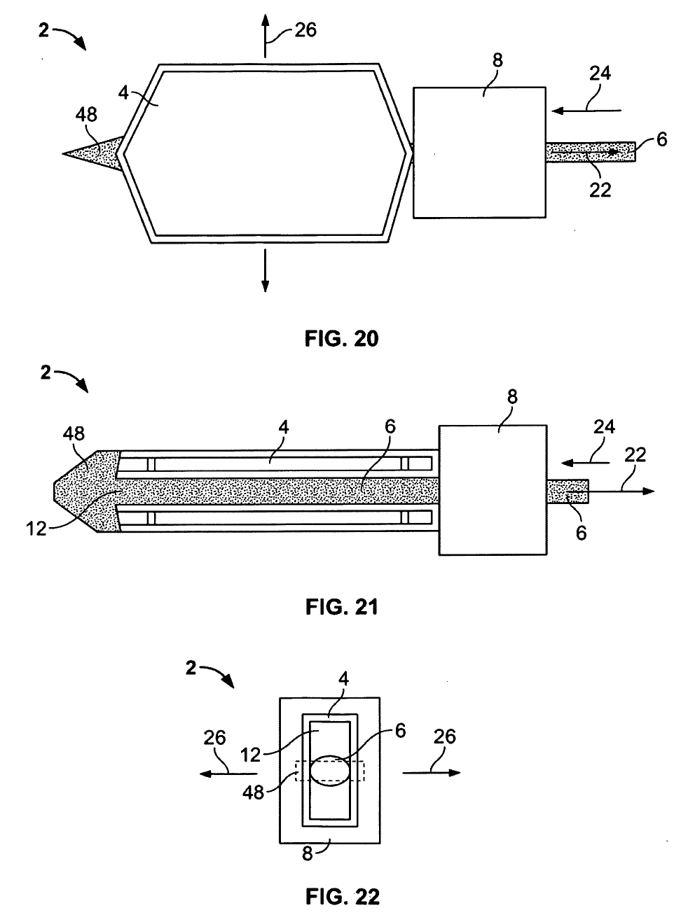

FIGS. 18 and 19 and separately FIGS. 20 through 22 illustrate various embodiments of the deployment system 2 that can be in a second, longitudinally compressed, configuration.

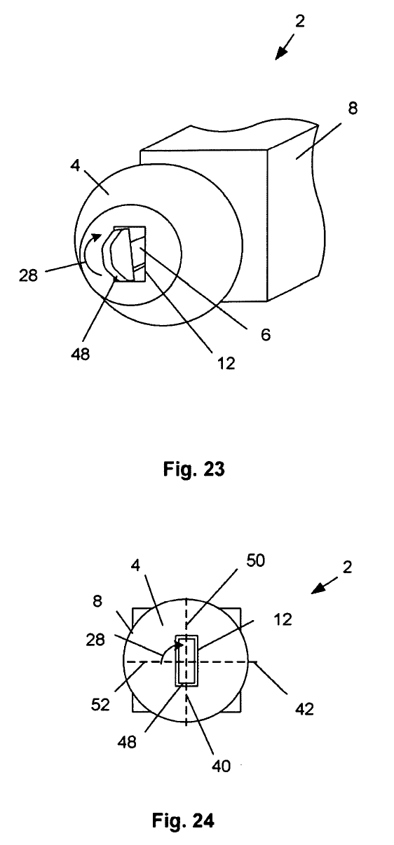

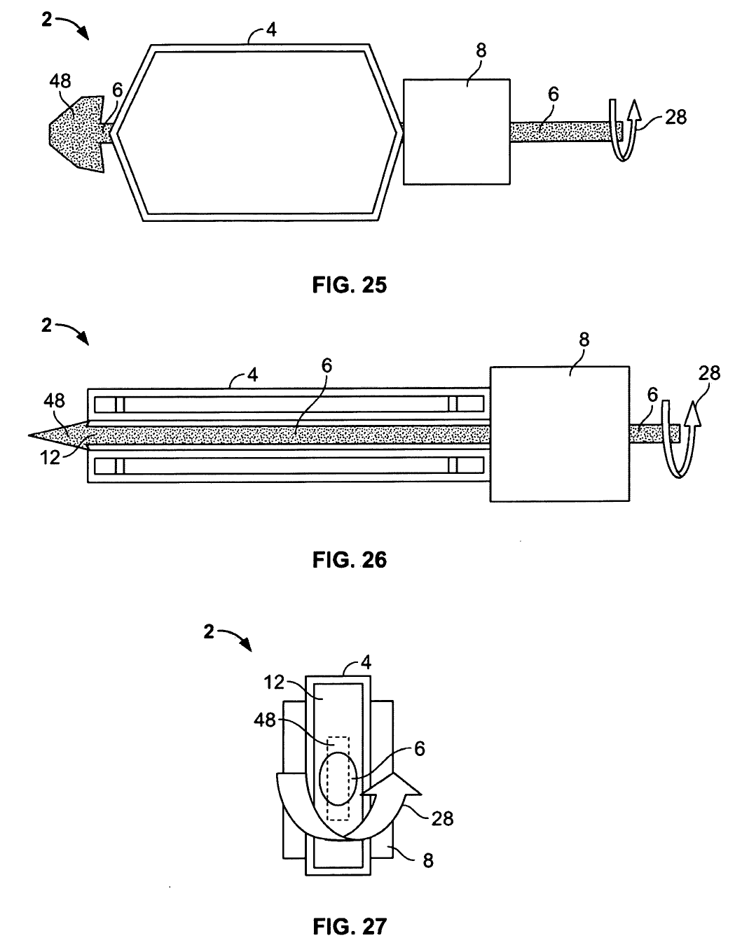

FIGS. 23 and 24 and separately FIGS. 25 through 27 illustrate that a rod rotation 28 torque, shown by arrow, can rotate (e.g., about 90 degrees) the rod 6 relative to the expandable support device 4. The rod rotation 28 torque can rotate the rod 6 relative to the expandable support device 4, for example, so that the port first axis 40 is substantially or completely aligned with the head first axis 50. The rod rotation 28 torque can rotate the rod 6 relative to the expandable support device 4, for example, so that the port second axis 42 is substantially or completely aligned with the head second axis 52. In the second (e.g., post-compression) configuration, the rod head 48 can be positioned to slidably translate within the expandable support device 4.

The rod 6 can have a non-circular configuration. The distal end port 12 can have a non-circular configuration. The rod 6 can transmit torque to the expandable support device 4 (e.g., at the distal end port 12), for example deforming the expandable support device 4 during deployment.

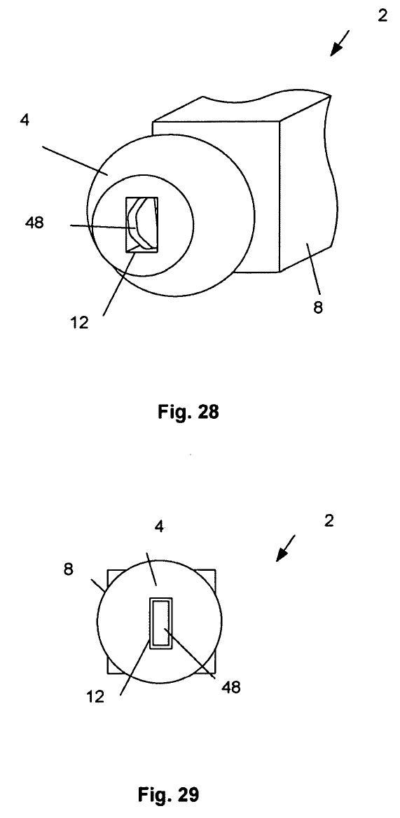

FIGS. 28 and 29 illustrate that the rod head 48 can be translatably withdrawn through the distal end port 12. The expandable support device 4 can be configured to separate from the anvil 8.

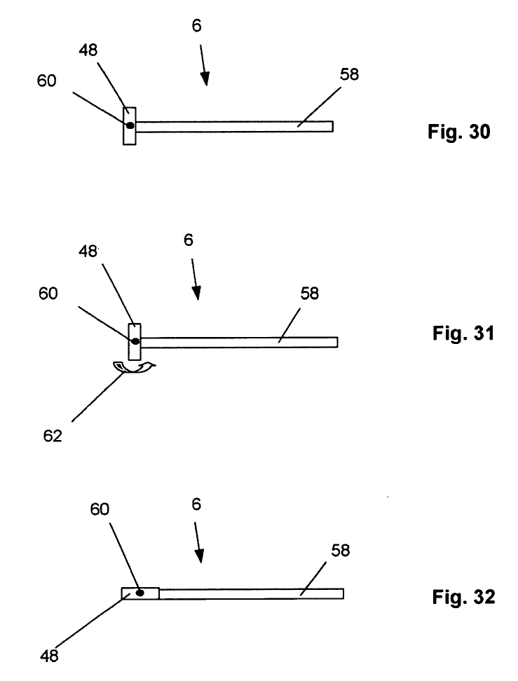

FIG. 30 illustrates that the rod 6 can have a rod body 58 and the rod head 48. In a first, (e.g., pre-compression and compression) rod configuration, the rod head 48 can be substantially or completely longitudinally perpendicular to the rod body 58. The rod 6 can have a rod hinge 60. The rod hinge 60 can rotatably attach the rod head 48 to the rod body 58.

FIG. 31 illustrates that a head rotation 62 torque. The head rotation 62 can rotate the head 48. FIG. 32 illustrates a second parallel rod configuration (e.g., post-compression) that can have the rod head 48 substantially or completely longitudinally parallel and/or aligned with the rod body 58.

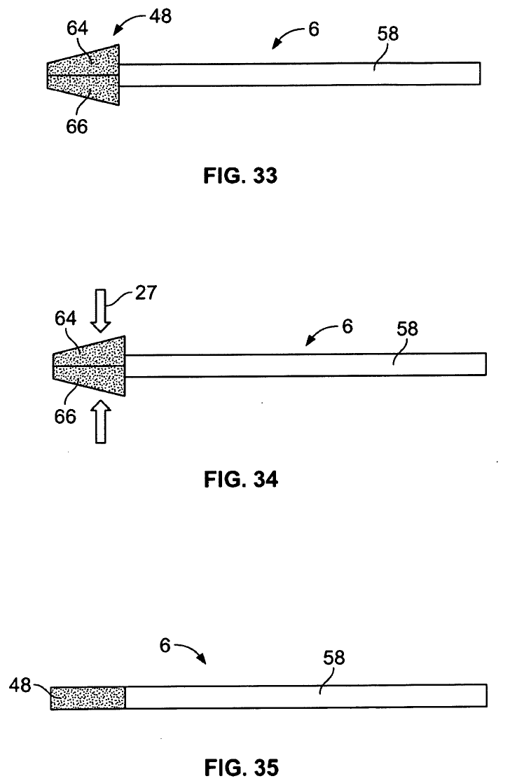

FIG. 33 illustrates that the rod head 48 can have one or more rod fingers such as a first rod finger 64 and a second rod finger 66. The rod fingers can extend radially beyond the rod body 58, for example in a first (e.g., pre-compression and compression) configuration. The rod fingers can be spring-loaded or otherwise resiliently biased to be in an extended configuration, as shown in FIG. 33.

FIG. 34 illustrates that the rod fingers can be radially compressed, as shown by arrows. FIG. 35 illustrates that the rod 6 can have a second (e.g., post-compression) configuration. The rod fingers can be configured to not radially extend beyond the rod body 58.

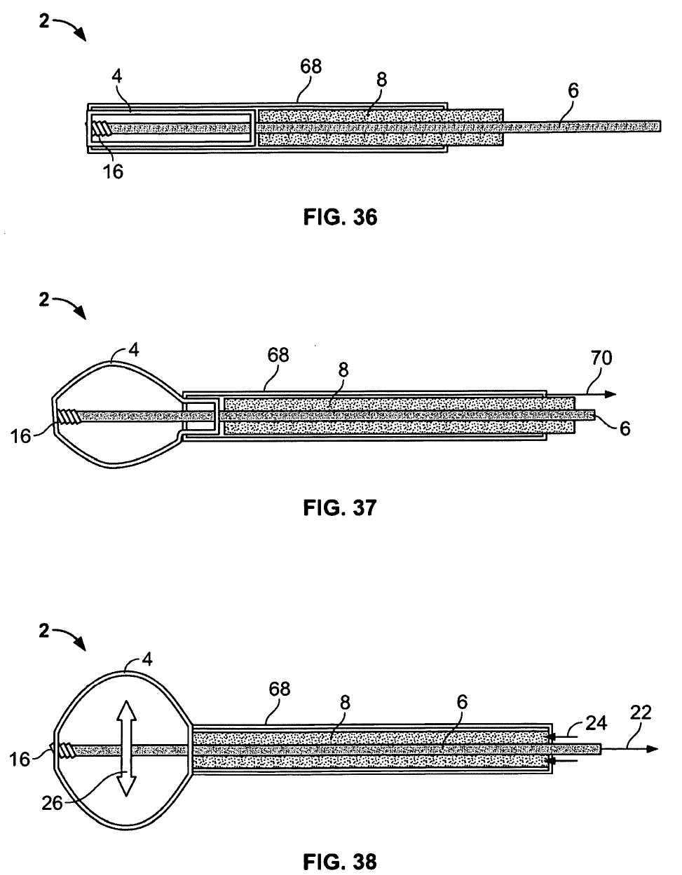

FIG. 36 illustrates that the deployment system 2 can have a sheath 68. In a first, (e.g., pre-compressed) configuration, the sheath 68 can be radially outside part or all of the expandable support device 4 and/or part or all of the anvil 8. The expandable support device 4 can be resiliently radially expandable and/or deformably radially expandable. The sheath 68 can prevent the expandable support device 4 from radially expanding 26.

FIG. 37 illustrates that the sheath 68 can be retracted from the expandable support device 4, for example by a sheath translation 70, shown by arrow. The sheath translation 70 can expose the expandable support device 4. The anvil 8 and/or rod 6 can force the expandable support device 4 from the sheath 68 (e.g., by transmitting a distally oriented force to the expandable support device 4). The anvil 8 and/or rod translation 72 is relative to the sheath 68. The sheath translation 70 is relative to the expandable support device 4.

FIG. 38 illustrates that the exposed expandable support device 4 can radially expand 26, as shown by arrows. The radial expansion 26 can be radial self-expansion and/or radial deformable expansion, for example, due to the anvil compression force 24, as shown by arrows, and rod compression force 22, as shown by arrow.

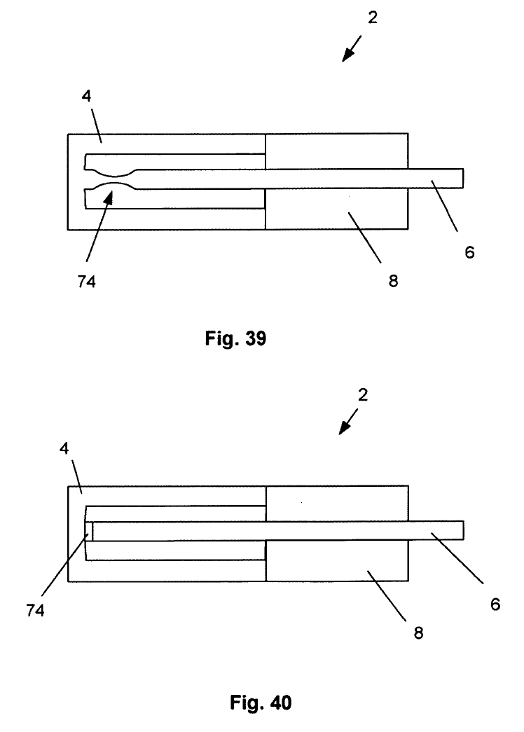

FIGS. 39 and 40 illustrate that the rod 6 can have a weakened zone 74. The weakened zone 74 can be a thinned portion of the rod 6, as shown in FIG. 39. As shown in FIG. 40, the weakened zone 74 can be made from a different material(s) (e.g., mechanically weaker, a lower thermal failure limit, different coefficient of thermal expansion, a layered combination of electrically conductive and resistive materials), including the same material in a different state (e.g., more porous, different heat treatment) than the remainder of the rod 6 and/or be separate from the remainder of the rod 6 and attached to the remainder of the rod 6 by an adhesive, weld, fusing (e.g., by heat and/or pressure), clip, snap, hook, hook and loop, friction fit, or combinations thereof. The weakened zone 74 can be in the middle of the rod 6 or at an end of the rod 6. The rod 6 can be attached to (e.g., as shown in FIG. 40) and/or integral with (e.g., as shown in FIG. 39) the expandable support device 4. The weakened zone 74 can be attached to and/or integral with the expandable support device 4.

FIG. 41 illustrates that the rod compression force 22 and anvil compression force 24 can be applied to the rod 6 and anvil 8, respectively. The expandable support device 4 can radially expand 26, as shown by arrows.

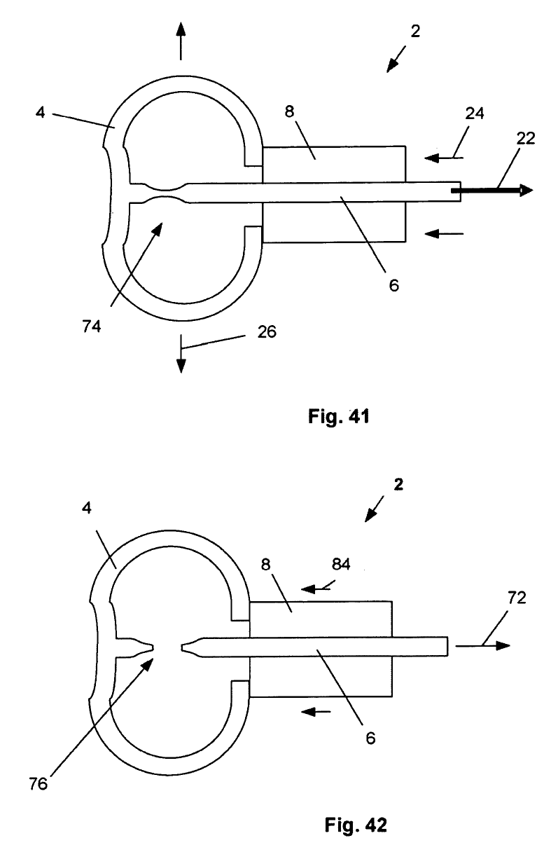

FIG. 42 illustrates that when the expandable support device 4 has radially expanded 26 to a designed-in expandable support device 4 radius, the weakened zone 74 can separate or fracture. The separation or fraction of the weakened zone 74 can be caused by, for example, tensile load failure of the weakened zone 74 and/or thermal (e.g., heat, cold, thermal shock) and/or electrical energy delivered, for example, along the rod 6. The separated or fractured weakened zone 74 can be a rod fracture 76. After fracture and/or separation, the rod 6 can be translated, as shown by arrow, relative to the anvil 8 translation, as shown by arrows. The expandable support device 4 can be deployed into a treatment site.

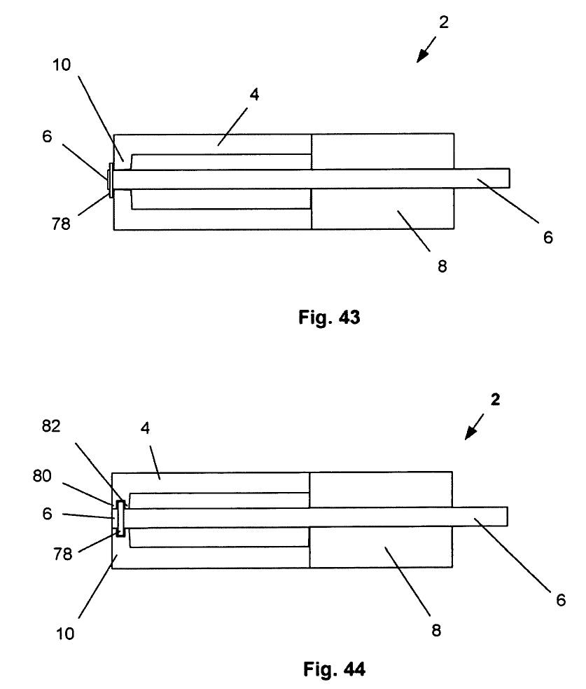

FIG. 43 illustrates that the rod 6 can have a pin 78. The pin 78 can extend radially beyond the rod 6 in one, two or more directions. In the first (e.g., pre-compression) system configuration, the pin 78 can be outside (e.g., distal to the distal end 10) of the expandable support device 4. The pin 78 can be separate and fixedly or rotatably attached to, and/or integral with, the rod 6. The pin 78 can be configured as an elongated, small-radius cylinder. The pin 78 can be configured as a substantially flat plate.

FIG. 44 illustrates that the expandable support device 4 can have a first catch 80 and/or a second catch 82. The first catch 80 and second catch 82 can be disposed on opposite sides (e.g., distal and proximal, respectively) of the pin 78.

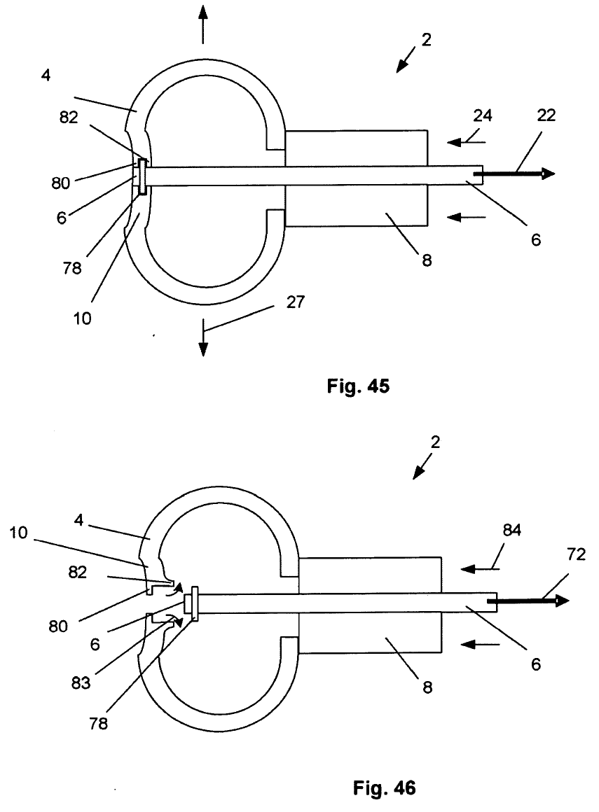

FIG. 45 illustrates that the rod compression force 22 and anvil compression force 24 can be applied to the rod 6 and anvil 8, respectively. The expandable support device 4 can radially expand, as shown by arrows 27.

FIG. 46 illustrates deploying the expandable support device 4 that can have a second catch 82 that can be weaker (e.g., structurally alterable more easily under mechanical stress than) the pin 78. The second catch 82 can be deformable or resilient. The second catch 82 can be malleable. When the expandable support device 4 has radially expanded to a designed-in expandable support device radius, the second catch 82 can rotate, as shown by arrows 83, for example, to release the pin 78. After the second catch 82 releases the pin 78, the rod 6 can be translated, as shown by arrow, relative to the anvil translation 84, as shown by arrows. The expandable support device 4 can be deployed into a treatment site.

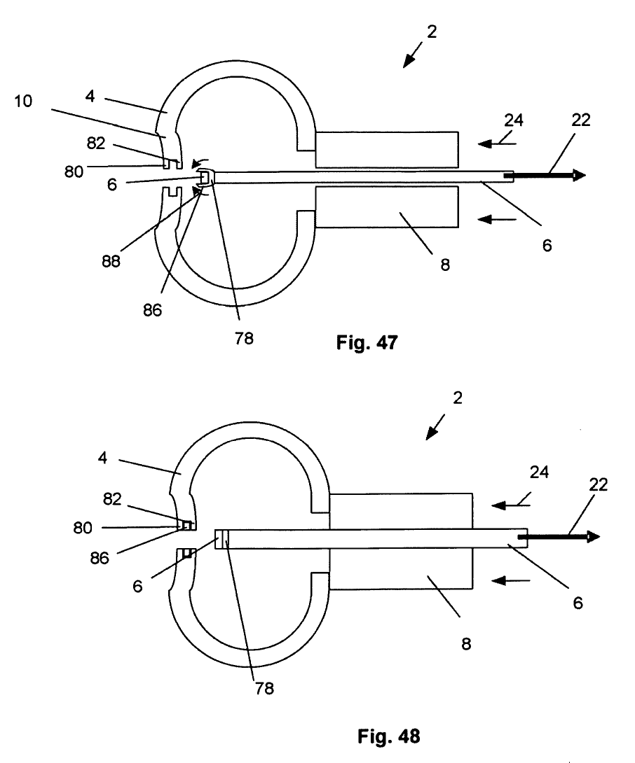

FIG. 47 illustrates deploying the expandable support device 4 that can have a pin 78 that can be weaker (e.g., structurally alterable more easily under mechanical stress than) the second catch 82. The pin 78 can be deformable or resilient. The pin 78 can be malleable. The pin 78 can have pin ends 86. The pin ends 86 can extend radially from the pin 78. The pin ends 86 can rotate 88 toward the pin 78, for example, to release from the second catch 82. The second catch 82 can rotate (as shown in FIG. 46) or not rotate. The pin ends 86 can rotate 88 enough to clear a gap between the second catch 82 and the rod 6.

FIG. 48 illustrates deploying the expandable support device 4 that can have a pin 78 that can be weaker (e.g., structurally alterable more easily under mechanical stress than) the second catch 82. The pin 78 can be deformable or resilient. The pin 78 can be brittle. The pin 78 can fracture. The second catch 82 can shear one, two or more pin ends from the remainder of the pin 78.

Any or all elements of the deployment system 2, including the expandable support device 4, and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON® from E.I. Du Pont de Nemours and Company, Wilmington, Del.), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high-performance polyethylene) fiber and/or yarn (e.g., SPECTRA® Fiber and SPECTRA® Guard, from Honeywell International, Inc., Morris Township, N.J., or DYNEEMA® from Royal DSM N.V., Heerlen, the Netherlands), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the deployment system 2, including the expandable support device 4, and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E.I. Du Pont de Nemours and Company, Wilmington, Del.), polypropylene, PTFE, ePTFE, nylon, extruded collagen, silicone or combinations thereof.

The deployment system 2, including the expandable support device 4, and/or elements of the deployment system 2, including the expandable support device 4, and/or other devices or apparatuses described herein and/or the fabric can be filled, coated, layered and/or otherwise made with and/or from cements, fillers, glues, and/or an agent delivery matrix known to one having ordinary skill in the art and/or a therapeutic and/or diagnostic agent. Any of these cements and/or fillers and/or glues can be osteogenic and osteoinductive growth factors.

Examples of such cements and/or fillers includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

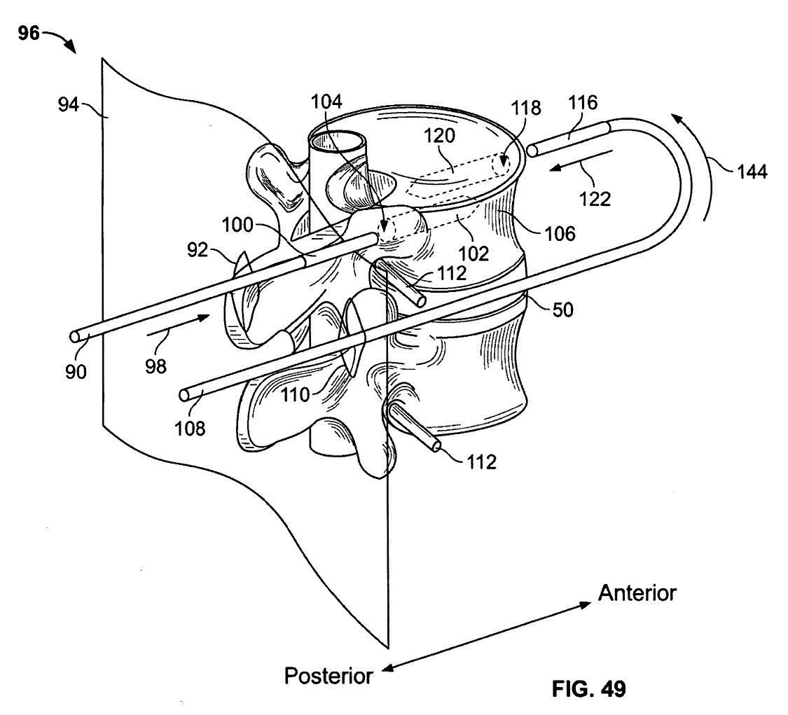

Method of Use FIG. 49 illustrates that a first deployment system 90 can enter through the subject's back. The first deployment system 90 can enter through a first incision 92 in skin 94 on the posterior side of the subject near the vertebral column 96. The first deployment system 90 can be translated, as shown by arrow 98, to position a first expandable support device 100 into a first damage site 102. The first access port 104 can be on the posterior side of the vertebra 106.

A second deployment system 108 can enter through a second incision 110 (as shown) in the skin 94 on the posterior or the first incision 92. The second deployment tool 108 can be translated through muscle (not shown), around nerves 112, and anterior of the vertebral column 96. The second deployment system 108 can be steerable. The second deployment system 108 can be steered, as shown by arrow 114, to align the distal tip of the second expandable support device 116 with a second access port 118 on a second damage site 120. The second access port 118 can face anteriorly. The second deployment system 108 can translate, as shown by arrow 122, to position the second expandable support device 116 in the second damage site 120.

The vertebra 106 can have multiple damage sites and expandable support devices deployed therein. The expandable support devices can be deployed from the anterior, posterior, both lateral, superior, inferior, any angle, or combinations of the directions thereof.

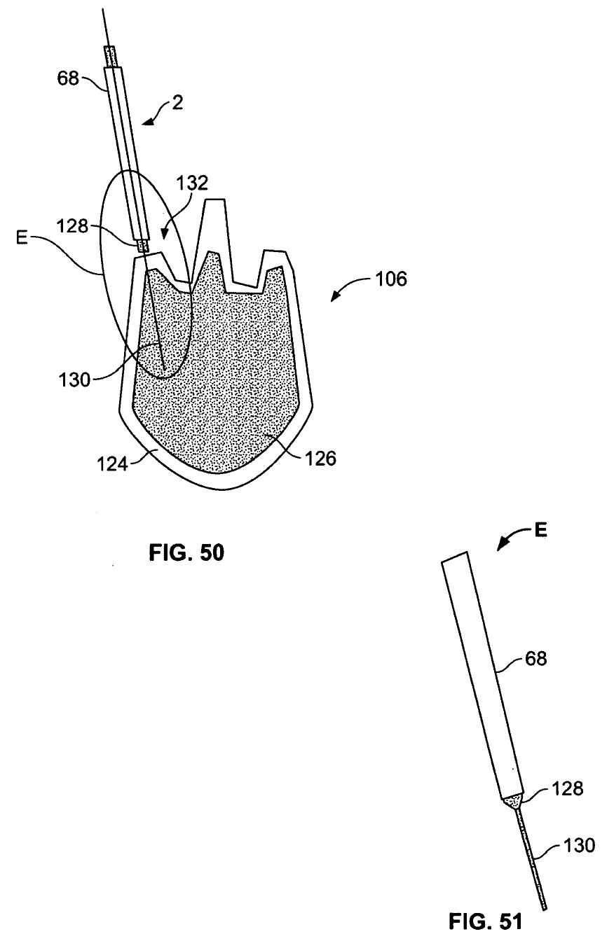

FIG. 50 illustrates that the vertebra 106 can have cortical (e.g., outer, harder, less porous and more dense) bone 124 and cancellous (e.g., inner, softer, more porous and less dense) bone 126. The deployment system 2 can have a drill 128 and/or a guidewire 130.

The deployment system 2 can be positioned adjacent to the vertebra 106, for example with the distal end of the deployment system 2 adjacent to a pedicle 132 of the vertebra 106. The guidewire 130 can be passed into the vertebra 106, for example into and/or through the cortical bone 124 and/or into the cancellous bone 126. The guidewire 130 can be passed through a pre-cut hole on the vertebra 106, and/or the guidewire 130 can be sufficiently rigid and sharp-tipped or screw-tipped to enter the vertebra 106 when a force is applied to the guidewire 130.

FIG. 51 illustrates that the drill 128 can be a rotary and/or vibratory and/or RF and/or acoustic drill. The drill 128 can be rigid, flexible, or combinations thereof (e.g., a proximal length of the drill 128 can be rigid and a distal length of the drill 128 can be flexible or vice versa). The drill 128 can be in the sheath 68. The drill 128 can be slidably or fixedly attached to the sheath 68. The drill 128 can have a channel or otherwise be cannulated. The drill channel can be radially centered in the drill 128 and extend along the longitudinal axis of the drill 128. The guidewire 130 can pass through the drill channel.

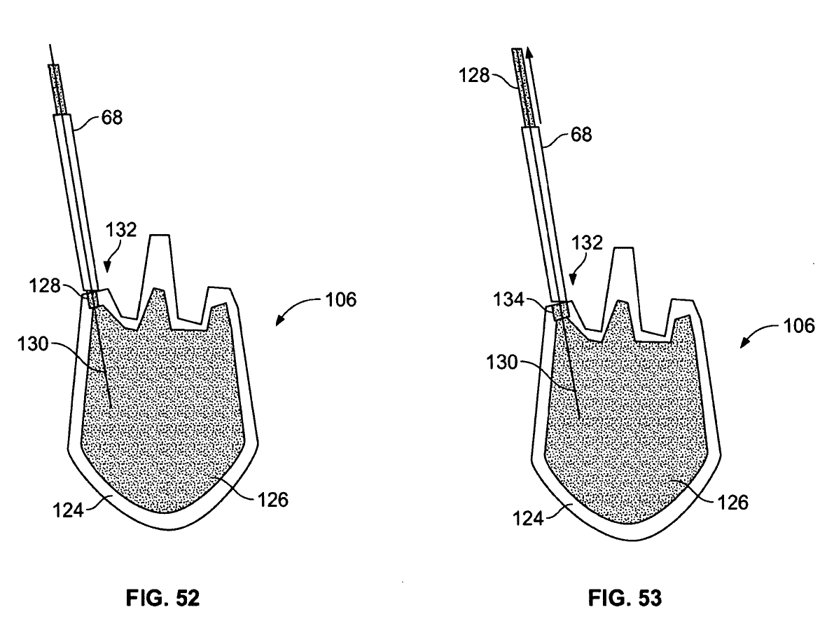

FIG. 52 illustrates that the drill 128 can be activated and pressed into the vertebra 106, for example at the pedicle 132. The drill 128 can drill into the cortical 124 and/or cancellous bone 126. For example, the drill 128 can drill from about 4.0 mm (0.16 in.) to about 5.0 mm (0.20 in.) deep into the vertebra 106, for example substantially in the transverse, or coronal, or sagittal plane. The tissue debris from drilling can be removed by suction delivered in the sheath 68.

FIG. 53 illustrates that the drill 128 can be translated, as shown by arrow, away from the vertebra 106. For example, the drill 128 can be removed from the sheath 68. The location where the volume of bone removed by the drill 128 previously resided can form a bone port 134.

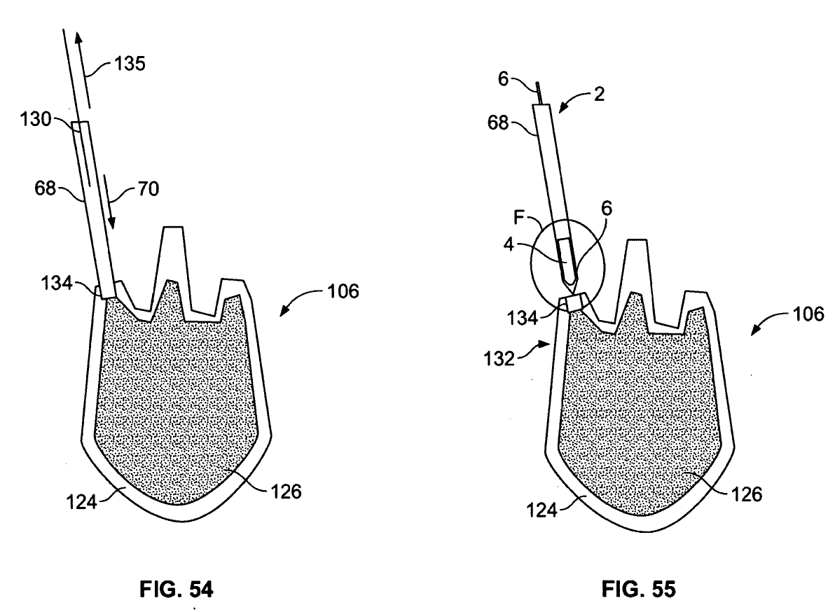

FIG. 54 illustrates that the sheath 68 can be translated, as shown by arrow, into the bone port 134. The sheath 68 can remain outside of the bone port 134. The guidewire 130 can be translated, as shown by arrow 135, out of the vertebra 106 and/or sheath 68, or the guidewire 130 can be left in the vertebra 106, for example to guide the expandable support device 4 and/or rod 6. The deployment system 2 can have a blunt distal end.

FIG. 55 illustrates that the expandable support device 4 and/or rod 6 can be placed adjacent to the vertebra 106, for example adjacent to the pedicle 132, for example adjacent to the bone port 134.

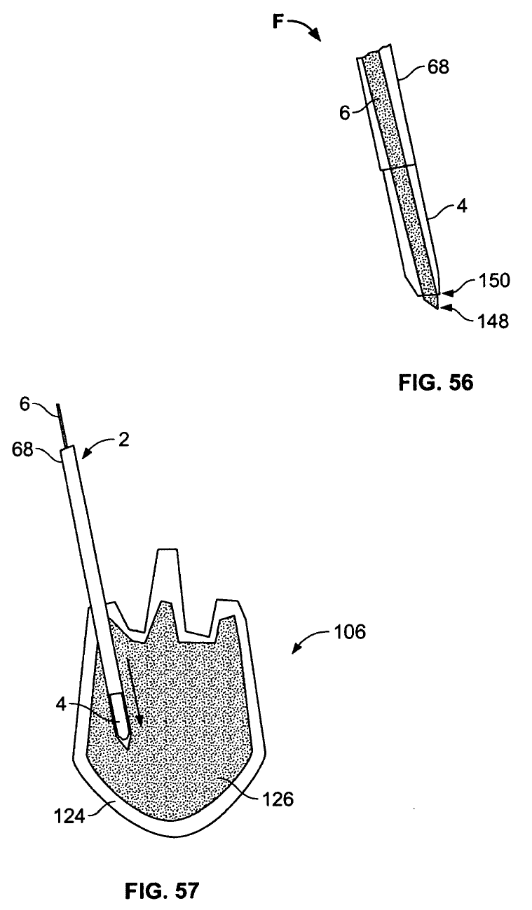

FIG. 56 illustrates that the deployment system 2 can have a dull or sharp deployment system distal end 148, for example, at the distal end of the rod 6. The expandable support device 4 can also have a dull or sharp expandable support device distal end 150. The deployment system distal end 148 and/or the expandable support device distal end 150 can be configured to compact and/or cut away bone during deployment, for example to translate through the bone (e.g., with less resistance) or to compress the bone (e.g., for improved fluid sealing at the deployment site).

FIG. 57 illustrates that the deployment system 2 can be translated, as shown by arrow, into the vertebra 106. The expandable support device 4 can be translated, as shown by arrow, into the vertebra 106, for example, into the cortical bone 124 and/or cancellous bone 126. The expandable support device 4 can be translated through the pedicle 132 (i.e., transpedicular) or around the pedicle 132 (i.e., extrapedicular).

During deployment, the target site can be visualized, for example with fluoroscopy, MRI, ultrasound, or combinations thereof.

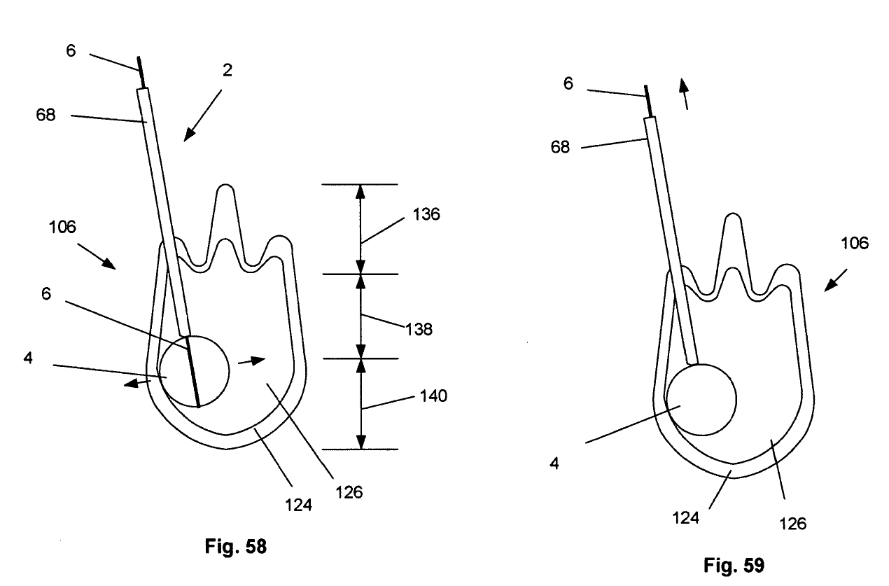

FIG. 58 illustrates that the deployment system 2 can be placed so that the expandable support device 4 can be in the posterior third 136 and/or medial third 138 and/or anterior third 140 of the vertebra 106. The expandable support device 4 can be located entirely within cancellous bone 126, entirely within cortical bone 124, or within bone cortical 124 and cancellous bone 126. The expandable support device 4 can be located entirely within the vertebra 106 or partially inside and partially outside the vertebra 106. The expandable support device 4 can be radially expanded, as shown by arrows, for example after being located in a desired position in the vertebra 106.

FIG. 59 illustrates that the rod 6 can be detached from the expandable support device 4 and translatably withdrawn, as shown by arrow, from the vertebra 106. The rod can be translatably withdrawn from the sheath 68.

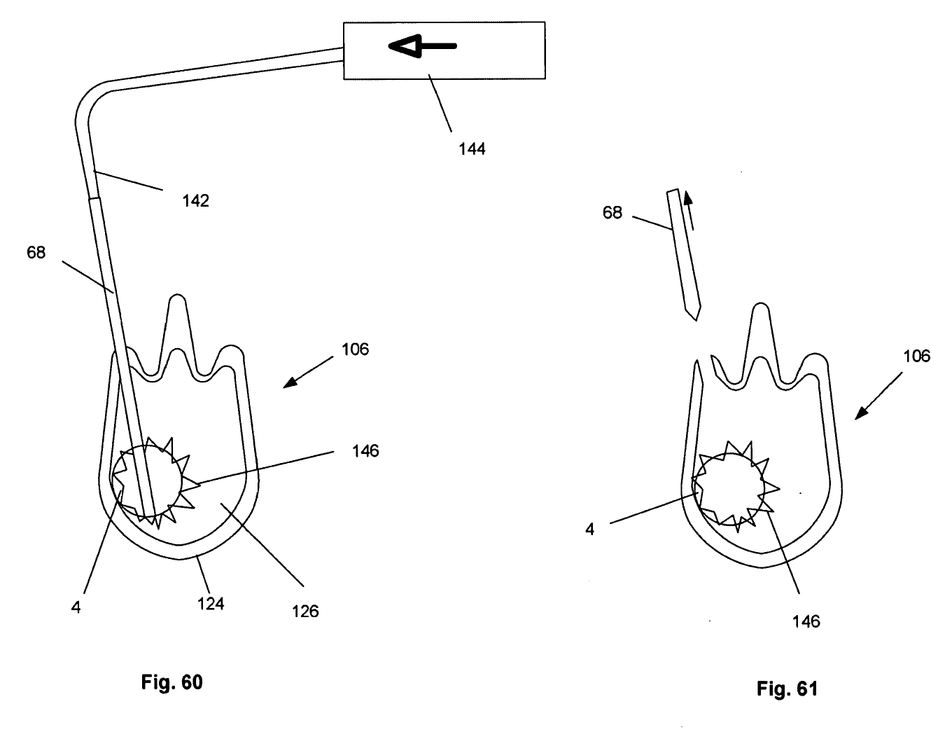

FIG. 60 illustrates that the sheath 67 can be attached to a filler conduit 142. The filler conduit 142 can be attached to a filler reservoir 144. The filler reservoir 144 can store and deliver a filler 146 under pressure, as shown by arrow. The filler reservoir 144 can be a refillable or replaceable cartridge or ampoule in the deployment system 2.

The filler 146 can be any material disclosed herein. For example, the filler 146 can be a cement, glue, agent, fabric (or single fibers), or combination thereof.

The filler 146 can be delivered (e.g., flow) through the filler conduit 142 and the sheath 68. The filler 146 can be deployed through the distal end of the sheath 68. The filler 146 can exit the sheath 68 at one or more distal ports 12. The distal ports 12 can be the port through which the expandable support device 4 is deployed and/or other ports, such as ports on the radial wall of the sheath 68.

The filler 146 can be configured to be deployed in a completely or partially liquid form. The filler 146 can be entirely or substantially solid (e.g., morselized bone). The filler 146 can be configured to solidify after delivery into the vertebra 106. The flow of the filler 146 can be substantially contained by the expandable support device 4 and/or the cancellous bone 126 and/or the cortical bone 124.

FIG. 61 illustrates that the sheath 68 can be withdrawn (e.g., rotated and/or translated), as shown by arrow, from the vertebra 106.

It is apparent to one skilled in the art that various changes and modifications can be made to this disclosure, and equivalents employed, without departing from the spirit and scope of the invention. Elements shown with any embodiment are exemplary for the specific embodiment and can be used on or in combination with any other embodiment within this disclosure.

We claim:

1. A deployment system for deploying one or more expandable support devices, the system comprising:
an expandable support device;
an elongate element having a first end and a longitudinal axis, wherein the first end of the elongate element is releasably attached to the expandable support device; and
an anvil, wherein the expandable support device abuts the anvil.

2. The system of claim 1, Wherein the anvil is longitudinally adjacent to the expandable support device.

3. The system of claim 1, wherein the first end of the elongate element is threadably attached to the expandable support device.

4. A deployment system for deploying one or more expandable support devices, the system comprising:
an expandable support device;
an elongate element having a first end and a longitudinal axis, wherein the first end of the elongate element is integral with the expandable support device; and
an anvil, wherein the expandable support device abuts the anvil.

5. The system of claim 4, wherein the elongate element is integral to the expandable support device at a failure region that breaks to release the expandable support device from the elongate element.

6. The system of claim 4, wherein the anvil is longitudinally adjacent to the expandable support device.

7. A method of deploying an expandable support device having a distal device end and a proximal device end using a deployment system having a first deployment element and a second deployment element, the method comprising:
releasably attaching the distal device end to the first deployment element,
forcing the proximal device end toward the distal device end,
detaching the distal device end from the first deployment element.

8. The method of claim 7, further comprising translating the expandable support device through bone to a target site before the forcing.

9. The method of claim 7, wherein the forcing comprises radially expanding the expandable support device.

10. The method of claim 7, further comprising delivering a filler to a target site, wherein the filler comprises a liquid.

11. The method of claim 7, wherein detaching comprises rotating the expandable support device with respect to the first deployment element.

12. The method of claim 7, wherein detaching comprises deforming the expandable support device.

13. The method of claim 7, wherein detaching comprises deforming the first deployment element.
